(19) United States
(12) Patent Application Publication
(10) Pub. No.: US 2002/0076754 A1
(43) Pub. Date: Jun. 20, 2002

(54) Overcoming AAV vector size limitation through viral DNA hetero-dimerization

(76) Inventors: Liangwu Sun, Pittsburgh, PA (US); Juan Li, Pittsburgh, PA (US); Xiao Xiao, Wexfoxd, PA (US)

Correspondence Address: KIRKPATRICK & LOCKHART LLP, 535 SMITHFIELD STREET, PITTSBURGH, PA, 15222, US

(21) Appl. No.: 09/838,786

(22) Filed: Apr. 20, 2001

Related U.S. Application Data
(60) Provisional application No. 60/198,673, filed on Apr. 20, 2000.

(51) Int. Cl. C12P 21/06

(52) U.S. Cl. 435/69.1

(57) ABSTRACT

A method for overcoming the packaging limitations of recombinant adeno-associated virus (AAV) particles through AAV heterodimer formation is provided. In the method, an expressed nucleic acid, typically a portion of a gene encoding a full-length therapeutic protein, or a functional derivative thereof, is split into two or more fragments by the insertion of one or more introns. Each intron is then split and each of the gene portions are inserted between AAV ITRs for packaging into recombinant adeno-associated virus particles. The recombinant viral particles are then co-infected into a target cell. Once inside the cell, the viral vectors form head-to-tail heterodimers through sequence homology of the inverted terminal repeats, thereby re-forming the intron. During mRNA maturation, the intron is spliced from the continuous DNA molecule, removing the intron and, thus, the intervening ITR sequences, thereby restoring the precise coding sequence of the expressed nucleic acid.

### CROSS-REFERENCE TO RELATED APPLICATIONS

This application claims priority under 35 U.S.C. § 119 (e) to U.S. application Ser. No. 60/198,673, filed Apr. 20, 2000.

### STATEMENT REGARDING FEDERALLY SPONSORED RESEARCH & DEVELOPMENT

This invention was made with government support under Grant Nos. R21 DK55966, AR 45967 and AR 45925, awarded by the National Institutes of Health. The government has certain rights in this invention.

### BACKGROUND OF THE INVENTION

1. Field of the Invention

The present invention is related to a method for overcoming the packaging limitations of recombinant adeno-associated virus (rAAV) particles through AAV heterodimer formation.

2. Related Art

Adeno-associated viruses (AAVs) are small non-enveloped single-stranded DNA viruses. AAVs are so named because their propagation is dependent upon co-infection by an unrelated virus, such as adenovirus or herpesvirusf for essential helper functions.

More than seven different types of AAV have been characterized. AAV type 1 (AAV1) through type 6 (AAV6) are all of primate origin. Avian AAV (AAAV) is from chickens and an ovine AAV was isolated from sheep. All of the primate AAVs, except AAV5, were originally isolated in laboratories as contaminants in adenovirus stocks. However, AAV5 was directly isolated from a human skin lesion as a herpes virus contaminant. Serological studies have showed that AAV2, AAV3 and AAV5 infections are common in humans, while AAV1 and AAV4 infections are prominent in monkeys. AAV6 appears to be a hybrid between AAV1 and AAV2, possibly arising from homologous recombination.

AAV is one of the smallest non-enveloped DNA viruses. The AAV viral particle is an icosahedron with a diameter of approximately 20-22 nm and a buoyant density of approximately 1.39-1.41 g mL-⁻¹ in a cesium chloride gradient. The AAV viral particle consists of approximately 26% DNA and 74% protein. The viral particle is very stable and resistant to certain proteases, repeated freezing and thawing, lyophilization, and temperatures up to 65° C. In addition, the particle also retains its structure and functions after exposure to a wide range of pH, such as a pH range from 3 to 9; to a number of detergents, such as deoxycholate; and to organic solvents, such as ether and chloroform.

Each AAV particle contains only one copy of the genome, which is packaged with equal frequency as either a positive strand or a negative strand. Both strands are equally infectious. The nucleotide sequences of the entire genome of all six primate AAVs have been determined and are available from GenBank (GenBank Accession Nos. NC_{—}002077 (AAV1), NC_{—}001401 (AAV2), NC_{—}001720 (AAV3), NC_{—}001863 (AAV3B), NC_{—}001829 (AAV4), AF085716 (AAV5) and NC_{—}001862 (AAV6)). The viral genome is approximately 4,700 nucleotides in length. The left portion of the AAV genome contains the Rep (replication) gene, which encodes the nonstructural proteins and is controlled by two separate promoters, p5 and p19. The right portion of the AAV genome contains the Cap (capsid) gene, which encodes the coat proteins and is controlled by the p40 promoter. Flanking the viral genome are two inverted terminal repeats (ITRs), which range in length from 140-170 nucleotides. For example, the extensively characterized ITR of AAV2 is 145 nucleotides.

ITRs are the only cis-acting sequences required for AAV DNA replication, packaging, chromosomal integration and provirus rescue. In addition, ITRs also exhibit residual promoter activities with unknown functions. However, ITRs are not required as a cis-acting element to achieve full-scale expression of viral genes in a productive life cycle. The left and the right ITRs on the single-stranded AAV genome can base pair with each other to form a double-stranded “panhandle” structure, with the remainder of the AAV DNA forming a large loop. This base pairing is essential for the repair of a damaged ITR, using the opposite ITR as a template. Each ITR can also form a T-shaped hairpin structure through its A/A′, B/B′ and C/C′ palindromic sequences. The ITR at the 3′ end of the viral genome serves as a primer for AAV DNA replication, while the nonpalindromic D sequence of the ITR contains a viral DNA packaging signal.

The AAV viral particle initially binds to and enters cells through a primary receptor, identified for AAV2 as the heparan sulfate proteoglycan receptor. In addition, a few co-receptors have also been identified, such as integrin αᵥβ₅ and fibroblast growth factor receptor. Upon entry into the cell, the viral particle migrates into the nucleus, uncoats and releases its viral DNA. The viral DNA then persists either as a free episome or as an integrated provirus in the host chromosomes. The specific integration is apparently mediated by the AAV Rep proteins.

AAV has two life cycles, latent infection and productive infection. In the absence of co-infection with a helper virus or genotoxic stress to the host cell, AAV establishes a latent infection in the host cells. On the other hand, with co-infection of a helper virus, AAV proceeds to the productive life cycle. Numerous unrelated viruses, such as adenovirus, herpes simplex virus, vaccinia virus and human papillomavirus, can all serve as a helper virus, although their effectiveness varies. The most commonly helper virus is adenovirus. In the productive life cycle, AAV's viral genes, Rep and Cap, are highly activated and expressed, leading to efficient viral DNA replication and packaging into progeny viral particles. Furthermore, latently infected AAV provirus can be activated and proceed to the productive life cycle by superinfection with a helper virus. Activation of a latent infection is termed “rescue.”

AAV employs integration as an essential step in its latent infection, integrating into the host chromosomal DNA as a provirus via its inverted terminal repeats. In addition, the proviruses often cluster in a head-to-tail tandem. AAV DNA integrates into the host chromosome in a site-specific manner. A number of AAV/cellular DNA junction sequences have been isolated from cell lines latently infected by wild-type AAV. The cellular junction DNA AAVS1 was localized on human chromosome 19 in the q13.3-qter region. Although the integration targets a specific region instead of a few nucleotides, the specificity to this region is apparently guided by a short sequence of chromosome 19 DNA that is similar to the consensus Rep-binding site on the A stem of the AAV ITR. The Rep78 or Rep68 proteins, but not the smaller Rep proteins or the capsid proteins, are required to mediate the targeted integration of AAV into the AAVS1 site on human chromosome 19 through the ITR sequence.

AAV is an attractive vector system for gene delivery and gene therapy. The foremost advantage of AAV as a virus-based vector is its safety. Recombinant AAV (rAAV) is the only viral vector system that is derived from nonpathogenic and replication-defective viruses. Also, AAV is not cytotoxic to its host cells. In addition, rAAV vectors have all the AAV DNA sequences removed except the ITRs, which are approximately 145 base pairs in length. The removal of all the viral genes adds another safety feature that prevents immune complications caused by undesirable viral gene expression. Another advantage of AAV is its broad host range and its ability to infect a wide variety of cells from different tissues, including muscle, brain, liver, lung, intestine and eye. The vectors can also be directly injected in vivo.

In addition, AAV is capable of infecting both dividing and nondividing (quiescent) cells. Furthermore, AAV vectors are capable of persistent gene transfer. The persistence is achieved by integration of the vector DNA into the host cell chromosomes. Alternatively, the vector DNA may persist as an episomal molecule without chromosomal integration. Moreover, the apparent lack of T-cell-mediated immune response against the transgenes in AAV vectors is another reason for sustained in vivo gene expression. The above features are appreciated for clinical gene therapy of a variety of genetic and acquired diseases.

Very high titers of rAAV particles can be produced, more than 10¹³ viral particles per milliliter. Currently, several methods are used for the production of AAV vectors. These methods share the same three essential components: a rAAV vector component, AAV Rep and Cap genes and helper functions provided by a helper virus. In the rAAV vector component, all of the AAV viral genes are removed and replaced by the foreign transgene(s), which is flanked by the AAV ITR sequences. The ITRs are the sole cis-elements required for vector DNA replication, packaging and integration. The AAV Rep and Cap genes provide trans-acting Rep and capsid proteins for vector DNA replication and packaging. However, these genes themselves can no longer be packaged into viral particles because they are physically separated from the ITRs and are located on a different DNA molecule or a different locus, such as a plasmid. The helper functions are derived from helper virus, such as adenovirus or herpes simplex virus. The helper virus facilitates efficient AAV gene expression, DNA replication and viral particle packaging.

When the above three components are introduced into a suitable host cell, for example, human 293 cells or HeLa cells, AAV Rep and Cap proteins are produced and the vector DNA is replicated and packaged into rAAV particles. The particles are released from the cell nucleus by physical or chemical means, such as sonication, freezing/thawing, or detergents. The virus particles can then be readily purified and concentrated according to their physical and biochemical properties, which are distinct from helper adenoviruses or herpes simplex virus. In addition to the classic CsCl gradient and nonionic iodixanol gradient centrifugation, heparin sulphate-based affinity chromatography is a particularly effective purification method of purifying rAAV particles. The availability of the high-titer, high-purity rAAV particles is a significant convenience to potential clinical gene therapy practice.

A widely used AAV vector-production strategy is the helper virus-free triple-plasmid transfection method (Xiao, et al., “Production of High-Titer Recombinant Adeno-associated Virus Vectors in the Absence of Helper Adenovirus,” J. Virol. 72, 2224-2232 (1998), which is incorporated herein by reference in its entirety). That method employs an AAV vector plasmid containing a transgene flanked by ITRs, a packaging plasmid containing Rep/Cap genes, and a helper plasmid containing a few essential adenovirus helper genes. The three plasmids are co-transfected into host cells, such as human 293 cells, to generate high-titer AAV vectors completely free of helper adenovirus contamination. This method is versatile and productive.

An alternative AAV vector production method is the use of packaging cell lines that harbor both the AAV vector and the AAV Rep/Cap genes. After infection with wild-type adenovirus, the cell lines produce high-titer AAV vectors. Similarly, other packaging cell lines harboring only the AAV vector component, but infected with a helper herpes simplex virus that also contains the AAV Rep/Cap genes, can efficiently produce AAV vectors as well. While convenient for large-scale production, these methods inevitably generate pathogenic helper virus that contaminates the AAV vector stocks, even though the helper adenovirus or herpes simplex virus could be carefully removed during purification.

Although infection of proliferating cells has been previously well documented, the first solid evidence of infection of nondividing cells by AAV vectors came from a study in the brain. This study unequivocally demonstrated long-term gene transfer by AAV vectors in neuronal cells of adult rat brain. Therapeutic efficacy was achieved in the parkinsonian rat model after AAV vector injection (Kaplitt, M. G., et al. (1994), “Long-term Gene Expression and Phenotypic Correction Using Adeno-associated Virus Vectors in the Mammalian Brain,” Nature Genetics 8, 148-54, incorporated herein by reference in its entirety). Subsequently, efficient and long-term gene transfer of more than 1.5 years duration was demonstrated in mature striatal muscle, another terminally differentiated tissue with largely nondividing cells (Xiao, X., J. Li, and R. J. Samulski (1996), “Efficient Long-term Gene Transfer Into Muscle Tissue of Immunocompetent Mice by Adeno-associated Virus Vector,” Journal of Virology 70, 8098-108, incorporated herein by reference in its entirety). More importantly, that study showed that in vivo gene transfer that resulted in synthesis of a foreign protein, such as β-galactosidase from Escherichia coli, via AAV vectors into immunocompetent animals, did not cause destruction of the transduced cells by cytotoxic T lymphocytes. The lack of cellular immune response and persistence of AAV vector DNA support long-term gene transfer in vivo in a number of different tissues.

Nevertheless, AAV vectors have an intrinsic limitation: they are derived from one of the smallest DNA viruses. This size limitation constrains the packaging of genes larger than about 5 kb. For this reason, current AAV vectors preclude the use of large genes such as dystrophin (Duchenne muscular dystrophy), dysferlin (limb girdle muscular dystrophy 2B), and Factor VIII (hemophilia A).

Previous efforts to circumvent the problem of the size limitation imposed by AAV vectors have focused mainly on altering the candidate genes into “mini-expression cassettes” that are suitable for AAV packaging. These strategies have included decreasing the size of the candidate gene by deleting non-essential sequences, as well as using minimal promoters and small polyadenylation signals. Although these approaches have had mild success with the gene for cystic fibrosis transmembrane conductance regulator protein (CFTR), most large genes are not suited for this strategy. In addition, this technique can lead to loss of protein functionality. Moreover, the use of minimal promoters usually restricts the ability to regulate gene expression properly, thus compounding the problem of controlling the therapeutic protein.

U.S. Pat. No. 6,200,560, incorporated herein by reference in its entirety, describes the therapeutic transfer of two complementary rAAV vectors, carrying the Factor VIII heavy and light chain, respectively, to mice. The vectors each included a 5′ promoter/intron regulatory region operatively linked to the coding region of the respective Factor VIII gene portions. The introns were included to increase expression of the individual genes, not to cause plicing between RNA produced by the complementary vectors. The complementary vectors were transferred to the mice via portal vein injection and Factor VIII expression was detected in the plasma of the treated mice. This method does not utilize heterodimerization to produce an mRNA encoding the complete Factor VIII protein, but results in the production of separate heavy and light chains that assemble after translation. One negative aspect of this method is that if a cell is infected with one vector and not the complementary set, an immune response may be generated to the heavy or light chain that would not have been generated in response to the assembled Factor VIII heterodimer. Consequently, a patient will produce anti-Factor VIII antibodies, thereby negating any therapeutic effect.

A unique feature of AAV biology is called viral DNA dimerization. This means that two AAV vectors can dimerize via homologous recombination of their ITRs. Each AAV vector contains a coding sequence flanked by two ITR sequences. Therefore, two AAV vectors can dimerize in one of four ways: vector 1 (5′ to 3′) to vector 2 (5′ to 3′), vector 1 (5′ to 3′) to vector 2 (3′ to 5′), vector 1 (3′ to 5′) to vector 2 (3′ to 5′), and vector 1 (3′ to 5′) to vector 2 (5′ to 3′). The heterodimerization of two AAV vectors when forming concatamers of either integrated or episomal templates is described in: Xiao, S., Li, J. and Samulski, R. J., “Efficient long-term gene transfer into muscle tissue of immunocompetent mice by adeno-associated virus vector,” J. Virology, vol. 704, pp. 8098-8108 (1961); Kotin, R. M. and Berns, K. I., “Organization of adeno-associated virus DNA in latently infected Detroit 6 cells,” Virology, vol. 170, pp. 460-467 (1989); Cheung, A. K., Hoggan, M. D., Hauswirth, W. W. & Berns, K. I., “Integration of the adeno-associated virus genome into cellular DNA in latently infected human Detroit 6 cells,” J. Virology, vol. 33, pp. 739-748 (1980); McLaughlin, S. K., Collis, P., Hermonat, P. L. & Muzyczka, N., “Adeno-associated virus general transduction vectors: Analysis of proviral structures,” J. Virology, vol. 62, pp. 1963-1973 (1988); and Duan, D. et al., “Circular intermediates of recombinant adeno-associated virus have defined structural characteristics responsible for long-term episomal persistence in muscle tissue,” J. Virology, vol. 72, pp. 8568-8577 (1998), erratum, vol. 73, p. 861 (1999), which are incorporated herein by reference.

Recombinant AAV vectors harboring test genes such as LacZ are capable of achieving highly efficient and sustained gene transfer in the mature muscle of immuno-competent animals for more than 1.5 years without detectable toxicity. Vector integration into the host DNA and the lack of CTL response against transduced cells support the potential of rAAV vectors for treatment of genetic diseases. Recently, researchers have greatly improved their ability to generate high titer and high quality rAAV. Successes have been reported in using rAAV vectors to deliver numerous reporter genes as well as therapeutic genes for metabolic diseases, including producing secreted therapeutic proteins using muscle and liver as a platform.

A method is therefore desired that allows the use of rAAV particles in therapies that require the transfer of nucleotide sequences of sizes greater than the packaging limits of the AAV vector.

### SUMMARY

A method is provided for overcoming the packaging limitations of recombinant adeno-associated virus particles through AAV heterodimer formation. The method of the present invention involves preparing two or more recombinant adeno-associated virus particles carrying different complementary portions of an expressed sequence and complementary portions of an intron. By co-infecting the particles into a cell, the recombinant vectors within the particles form heterodimers and produce a mature, functional MRNA and, ultimately a protein.

In one embodiment, an expressed sequence, typically a sequence encoding a desired protein that is operably linked to a suitable promoter and a polyadenylation sequence is provided. Next, an intron is inserted into the expressed sequence so that the expressed sequence is split into fragments that will fit into an AAV viral particle. The intron is then split between its 5′ junction and its splice acceptor sequence. The nucleic acid fragments of the spliced gene are then individually packaged into AAV vectors, flanked by two AAV inverted terminal repeat sequences.

The viral particles produced by this method, containing the split gene fragments, may be co-infected into a cell, typically for therapeutic purposes, so that the AAV vectors form heterodimers. After heterodimer formation, the dimerized nucleic acid is transcribed to produce pre-mRNA. The pre-mRNA intron, containing the consensus intron sequences as well as a single ITR resulting from the heterodimerization process, is then spliced from the pre-mRNA by cellular spliceosome machinery. This results in the precise restoration of the split coding sequence in the newly formed mRNA. Afterwards, the mRNA is translated into a functional protein.

In addition to the above embodiment wherein the gene is separated into two parts (a two component system), the method of the invention also includes embodiments wherein a gene is separated into three parts. The three-component system requires an extra intron and an extra viral particle.

Also provided is a recombinant adeno-associated virus particle. The particles include a rAAV vector including a portion of an expressed sequence that is flanked by two adeno-associated virus inverted terminal repeat sequences. The rAAV vector includes one of three nucleic acid fragments: a promoter operably linked to a first expressed nucleic acid having at its 3′ end a 5′ intron splice junction; a second expressed nucleic acid operably linked to a polyadenylation sequence and having at its 5′ end a 3′ intron splice junction operably linked to a splice acceptor sequence; and an optional third expressed “middle” nucleic acid having at its 5′ end a 3′ intron splice junction operably linked to a splice acceptor sequence, and at its 3′ end a 5′ intron splice junction. The rAAV particles each include a portion of an expressed sequence that, when combined as a complementary set, will produce a complete mRNA.

Also provided is a method for producing a spliced ribonucleic acid and/or a protein. The method includes transducing a cell capable of transduction by a rAAV particle with a complementary set of the above-described complementary rAAV vectors.

Further aspects of the invention include a pharmaceutical composition and nucleic acid vectors. Each of these embodiments include either the above-described vectors or rAAV particles.

Other details, objects and advantages of the present invention will become apparent with the following description of the present invention.

### BRIEF DESCRIPTION OF THE DRAWINGS

The following drawings are for illustrative purposes, to facilitate better understanding of the invention and not for limiting the same.

FIG. 1 shows consensus sequences around 5′ and 3′ splice sites in vertebrate pre-mRNA (Lodish, H. at al., Molecular Cell Biology, 4^{th} ed., W. H. Freeman & Co. 2000, p. 416).

FIG. 2 illustrates schematically the strategy for packaging a large gene by the split AAV vectors through heterodimerization.

FIGS. 3A-E show X-gal staining of the human 293 cells 4 days after infection with AAVLacZ-5′ split vector alone (3a), infection with AAVLacZ-3′ split vector along (3b), co-transfection with pAAVLacZ-5′ and pAAVLacZ-3′ plasmids (3e), showing very few scattered blue cells), infection with AAV-LacZ that contained an intact gene expression cassette (3d), co-infection with AAVLacZ-5′ and AAVLacZ-3′ split vectors (3e). FIG. 3F shows staining of mouse gastrocnemius muscle 4 months after injection with a 50-μl mixture of AAVLacZ-5′ and AAVLacZ-3′ vectors (5×10¹⁰ total viral DNA particles) . FIG. 3g is a bar graph showing β-galactosidase enzyme activity in 293 cells 4 days after infection with AAV viral vectors at a multiplicity of infection of 5×10³ viral DNA particles/cell. Data represent six experiments (n=6).

FIGS. 4A-D. Characterization of the split AAV vector heterodimerization by RT-PCR and DNA-PCR. FIG. 4A shows viral heterodimer junctions and restriction enzyme digestion map. AAV ITR can exist in two different orientations (‘flip’ or ‘flop’). 5′-LacZ and 3′-LacZ are the split LacZ coding regions. 5′ intron and 3′ intron are the split hCG intron sequence; PCR-F and PCR-R are the two primers to amplify the heterodimeric DNA or its mRNA after reverse transcription. FIG. 4B shows gel electrophoresis of RT-PCR products after amplification of mRNA from cells co-infected with split vectors. CMV-LacZ (positive control) contains the original LacZ coding sequence without the intron. RT: with (+) or without (−) reverse transcriptase. FIG. 4C shows gel electrophoresis of DNA PCR products after amplification of ‘Hirt DNA’ from cells co-infected with split vectors. Plasmid CMV-LacZ-Int (control) lacks the ITR insertion in the hCG intron. FIG. 4D shows a restriction digestion analysis of the 1.0-kb heterodimer junction PCR product which was gel-purified from lane 3 of FIG. 4C. SmaI digestion within the ITR generates four bands, with two form each orientation, ‘flip’ and ‘flop’ (see FIG. 4A).

### DETAILED DESCRIPTION

Described herein are methods for overcoming the packaging limitations of recombinant adeno-associated virus (rAAV) particles through AAV heterodimer formation. By this method, expressed sequences, such as protein coding sequences, larger than the approximately 5 Kbp packaging limit of rAAV particles, can be transferred to cells by rAAV particles. The method exploits AAV viral DNA dimerization, a unique feature of AAV biology, and intron splicing methods to rejoin the split gene in the correct reading frame. The method offers great hope in the area of gene transfer and gene therapy.

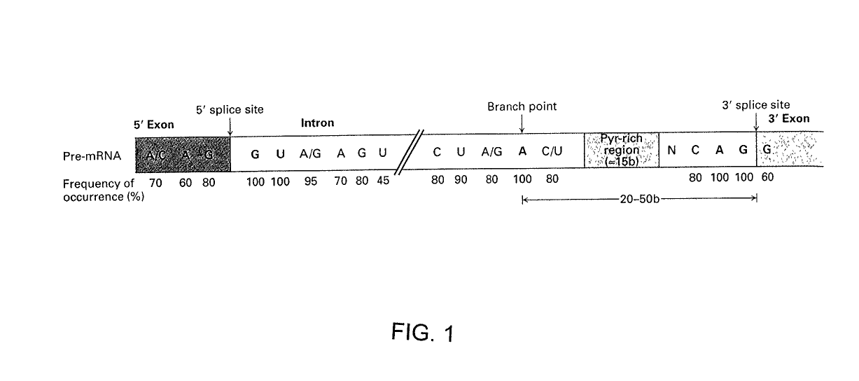

Maturation of pre-mRNA into mRNA is a very well characterized biological process. In the formation of mRNA from pre-MRNA, introns are removed and exons are spliced together at exon-intron splice junctions. Analysis of a large number of mRNAs has revealed that certain nucleotides are conserved in typical introns and splice junctions, as shown in FIG. 1. For example, nearly invariant bases of an intron are the 5′-GU and the 3′-AG. Certain bases that flank these 5′ and 3′ conserved regions often are found in abnormal (non-random) frequencies. Also conserved is the branch-point adenosine, usually 20-50 bases from the 3′ splice site. However, the central region of the intron, which may range from 40 to 50,000 bases in length, is generally unnecessary for splicing to occur. Introns are removed from RNA or pre-mRNA as a lariat structure by spliceosomes. The splicing together of exons proceeds via two sequential transesterification reactions.

The present system is robust in its reliance on a combination of standard cellular machinery and well-characterized rAAV biology and well-established rAAV techniques. Most, if not all, pre-mRNAs will splice in vitro and in vivo if they contain consensus intron sequences. Many laboratories have demonstrated the ability of rAAV vectors to provide therapeutic quantities of proteins in vivo. It also is known that AAV vectors will heterodimerize in vivo. As shown herein, the transfer of complementary rAAV vectors for LacZ and Factor VIII in vitro and in vitro results in production of functional protein. Therefore, so long as the dual vectors can be synthesized, by standard cloning methodologies, rAAV heterodimers should form and the transcribed pre-MRNA should splice; resulting in production of a desired protein product.

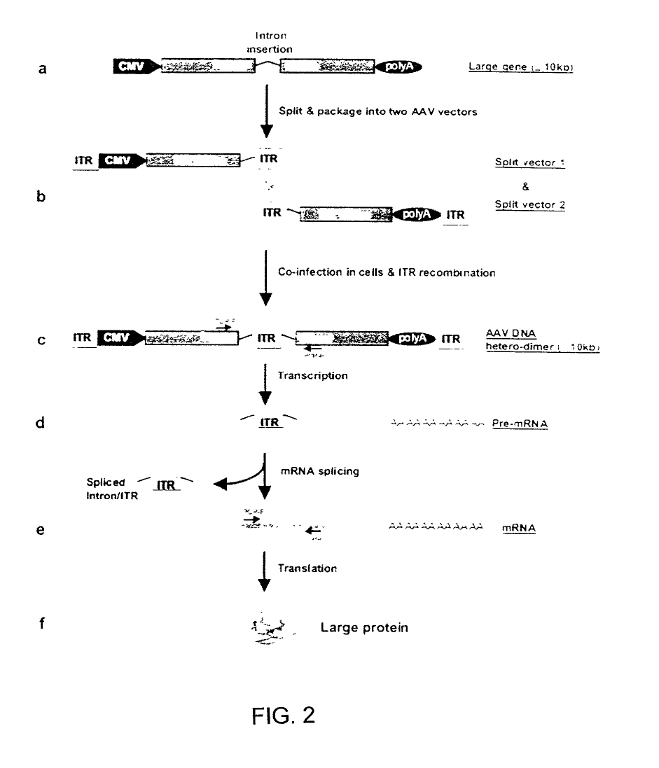

Provided is a method for preparing split rAAV particles and co-infecting the particles into a cell so that the recombinant AAV nucleic acids of the particles can form heterodimers and produce a functional, spliced mRNA. An embodiment of the method of the invention utilizing two recombinant adeno-associated viral particles to ultimately form a large protein is illustrated in FIG. 2.

The first step of the method involves preparing recombinant adeno-associated virus particles, as illustrated in FIG. 2. This involves providing a nucleic acid containing two adeno-associated virus inverted terminal repeat sequences that flank a portion of a gene to be expressed. The portion of a gene to be expressed (the “expressed sequence”) typically encodes a portion of protein product but can produce a functional RNA, if desired. The expressed sequence typically is divided into two or more portions, most typically in half, and the two or more portions each are inserted between two operational ITR sequences to produce two or more rAAV constructs. Each portion of the expressed sequence is operably linked to a suitable regulatory elements and portion of an intron.

For instance, a 7 Kbp nucleic acid encoding a protein product may be cut in half, resulting in a 3.5 Kbp 5′ coding sequence and a 3.5 Kbp 3′ coding sequence. A promoter, such as one of the RSV-LTR or CMV promoters, may be operatively linked to the 5′ end of the 5′ coding sequence to make a 5′ construct. A polyadenylation signal sequence (“poly (A) sequence”) may be operatively linked to the 3′ end of the 3′ coding sequence to make a 3′ construct. A 5′ splice junction may be added to the 3′ end of the 5′ coding sequence and a 3′ splice junction, operatively linked to a splice acceptor sequence, may be added to the 5′ end of the 3′ coding sequence. These 5′ and 3′ constructs are then inserted between AAV ITR and rAAV particles are produced. As such, in use, when the packaged 5′ and 3′ vectors are used to transduce a single cell, heterodimers will form. When heterodimerization occurs with the 5′ construct upstream (5′) to the 3′ construct and in a head-to-tail orientation, a pre-mRNA is produced containing both the 5′ and 3′ coding sequences separated by a functional intron. Within the intron is the heterodimer junction, including an ITR sequence. The intron is then excised by ubiquitous cellular machinery (spliceosomes), resulting in an mRNA encoding the entire protein product.

The recombinant AAV constructs may be prepared or synthesized by any of the many recombinant methods known in the art, a few of which are described hereinbelow.

As used herein, a “gene” is an operative genetic determinant in its broadest sense. Typically, a gene encodes a protein or RNA product. A typical gene is a nucleic acid that includes a coding region for a protein, along with operably linked regulatory sequences, including, but not limited to, promoters, enhancers, terminators and polyadenylation (polyA) sequences. Promoters can be, for example and without limitation, constitutive or semi-constitutive (i.e., CMV and RSV promoters) or tissue-specific promoters (i.e., a muscle creatinine kinase (MCK) promoter). The term “expression” or “gene expression,” and like words and phrases, mean the overall process by which the information encoded on a gene or nucleic acid is converted into a ribonucleic acid and/or a protein, or a post-translationally modified version thereof, and/or an observable phenotype.

The expressed sequence may be synthesized and/or isolated from, for instance and without limitation, a genomic DNA library, CDNA library, vector, plasmid, cosmid, phage or any other gene source known in the art by any method, for instance, by PCR. The expressed sequence may also be of any origin. Genes of human origin may be preferred in some instances. The gene may code for any one of a number of proteins, but typically, the protein product of the gene will have potential therapeutic value. For instance, the gene may be the dystrophin gene, the Factor VIII gene or the gene encoding cholesterol transport protein (ABC1).

Other genes that are well characterized and that are particularly suitable for use in the methods and compositions described herein are the Factor VIII, dysferlin and ATP binding cassette transporter genes. Defects in the Factor VIII gene product (GenBank Accession No. E00527) are found in patients with hemophilia A. Defects in the dysferlin gene product (GenBank Accession No. NM_{—}003494) are found in patients with LGMD 2B and Miyoshi myopathy. Defects in the ATP binding cassette transporter gene product (GenBank Accession No. NM_{—}007168) are linked to defects in cholesterol metabolism, and therefore, heart disease.

In one method, once an expressed sequence is isolated, an intron is inserted into the expressed sequence. As mentioned above, the point of insertion may split the expressed sequence in roughly equal halves. However, this is not necessary and may be undesirable where large promoters or other regulatory sequences are used. As used herein, an “intron” is broadly defined as a sequence of nucleotides that is removable by RNA splicing. “RNA splicing” means the excision of introns from a pre-mRNA to form a mature mRNA. Insertion of an intron into an expressed sequence can be accomplished by any method known in the art. The only limitation of where the intron is inserted, is in consideration of the packaging limitations of the AAV virus particles (about 5 kbp).

The typical intron contains a 5′ splice site or junction, a splice acceptor or branch point, and a 3′ splice site or splice junction. The term “5′ splice site” or “5′ splice junction” means the location of the exon-intron junction wherein the junction is between the 3′ end of the 5′ fragment of a gene or nucleic acid fragment and the 5′ end of the intron, and includes the consensus sequence at the 5′ end of the intron that is required for RNA splicing. The term “splice acceptor” or “branch point” refers to the nucleotide, usually adenosine, located approximately 20-50 bp from the 3′ splice site that helps form the lariat structure during the first trans-esterification reaction during RNA splicing. The term “3′ splice site” or “3′ splice junction” means the location of the exon-intron junction wherein the junction is between the 5′ end of the 3′ fragment of a gene or nucleic acid fragment and the 3′ end of the intron, and also includes the consensus sequence at the 3′ end of the intron that is required for RNA splicing. The term “consensus sequence” means the nucleotides in/or adjacent to either the 5′ or 3′ splice junction that are required for RNA splicing; these sequences are usually either invariant or highly conserved.

As illustrated in FIG. 2, an intron typically is inserted into a gene of less than 10 kb at approximately its midpoint so that the two fragments will be less than the AAV packaging limit; the first fragment containing a promoter and the 5′ end of the gene and the second fragment containing the 3′ end of the gene and a polyA tail. The intron may be derived from any source, such as from a genomic library. An intron may be obtained by polymerase chain reaction (PCR) from human DNA using primers, as described below. Any intron capable of RNA splicing in cells to be transduced by the rAAV particles can be used in the method of the present invention. In reference to the typical vertebrate intron consensus sequences, so long as consensus splice junction and splice acceptor sequences are engineered into the construct, the methods used to obtain and insert those sequences and the source of those sequences are immaterial. FIG. 1 shows intron consensus sequences for a typical mammalian intron. In the example below, the intron is the human chorionic gonadotropin (hCG) gene 6 intron 1.

As a next step in the production of rAAV virus particles according to this embodiment of the present invention, the intron is split between the 5′ splice junction and the splice acceptor in order to physically separate the gene into 5′ and 3′ constructs.

After splitting the gene into 5′ and 3′ constructs, the constructs are inserted between AAV ITR sequences in AAV vectors on separate plasmid backbones. Typically, the ITRs are the same for both vectors. Any AAV cloning vector may be used to prepare the rAAV vectors described herein, such as pSSV9 and PXX-UF1 described herein. Packaging of the rAAV vectors is performed according to standard protocols, as described herein.

After preparing the two complimentary rAAV virus particles, the virus particles containing the various parts of the split gene are co-infected into a cell, permitting the vectors within the particles can form heterodimers. The virus particles may be delivered in vivo by a number of routes, but typically intramuscularly and into the portal vein for delivery to the liver. Any amount of virus particles may be delivered, so long as the multiplicity of infection is sufficiently high to ensure delivery of both complementary particles to a sufficient number of cells to create a therapeutic effect. Recombinant AAV particles can be produced to a very high titer (at least 10¹³ particles per ml) so that intramuscular delivery, or even portal vein delivery can deliver therapeutic quantities of the virus to cells.

The virus particles prepared as described above may be formulated as a pharmaceutical composition containing a sufficient amount of the particles to transduce a desired number of targeted cells. The pharmaceutical composition comprises virus particles made according to the present invention and a pharmaceutically acceptable excipient. The excipient may facilitate or enhance delivery of the virus particles to a patient or transduction of a patient's cells. Specific examples of excipients include, without limitation, buffers, salts, adjuvants, proteins or peptides, polymeric materials, dyes, monosaccharides, disaccharides and polysaccharides. The excipient may be phosphate buffered saline, or similar buffer systems, such as Tris-EDTA-NaCl (TES).

One preferred site of delivery of rAAV particles is muscle cells or muscle tissue. Muscle cells and/or tissue include all types of muscle cells and progenitors thereof. Examples of administration and expression of rAAV particles in muscle cells can be found in PCT Publication WO/9640272, U.S. Pat. No. 5,858,351, both of which are incorporated herein by reference, and Li et al., 1999). Generally, skeletal muscle is a preferred site for administration of rAAV particles because it is not a vital organ and it is easy to access. When the protein to be expressed is dystrophin, it is preferred that the rAAV particles are administered to muscle.

Use of muscle-specific promoters, such as the muscle creatine kinase promoter, (MCK), as opposed to semi or fully constitutive promoters, such as the CMV and RSV promoters, is preferred when the rAAV is administered intramuscularly to target expression of the protein to muscle cells and tissue and to prevent expression in other tissues, or in the host cell in which the recombinant virus particles are prepared, which in the case of the dystrophin gene, and most likely in other protein delivery systems, would be undesirable. In the case of delivery of dystrophin by rAAV particles, rAAV may be administered to each muscle or systemically. If the promoter were not muscle-specific, it is likely that this broad dissemination of rAAV would result in transformation of a large number of non-muscle cells in which expression of the dystrophin gene is not desirable and might be harmful. The MCK promoter is 1.2 kbp in size and smaller versions of this promoter have become available. Since dystrophin can be broken into two 3.5 kbp fragments, the two resultant genes would fall within the 5 kbp packaging limitations of rAAV particles.

### EXAMPLE 1

Split AAV Vectors (SAVE) With LacZ Gene

To assess the utility of generating heterodimer vectors for expressing large genes, the gene for β-galactosidase (LacZ) in combination with the gene for human chronic gonadotropin (hCG) (ref. 20) intron 1 was used as a model system. LacZ is a relatively large reporter gene (3.2 kb) and is widely used for both colorimetric X-gal staining and quantitative β-galactosidase enzyme assays. PCR was used to generate approximately equal fragments of the LacZ coding sequence using pCMVβ as a template. FIG. 2 illustrates the general scheme for producing a protein product by the method described herein. In reference to FIG. 2, a large gene was inserted with an intron to break the gene cassette into two parts. The intron is the hCG gene 6 intron 1, which is obtained by PCR from human DNA using primers hCGln-F (5′-GTAAGACTGCAGGGCCCCTGGGCAC-3′ (SEQ ID NO: 1)) and hCGln-R (5′-CTGGGACAAGGACACTGCTTCACC-3′ (SEQ ID NO: 2)). A breakpoint that facilitated the engineering of a consensus exon-joining site (AG/N) was chosen for insertion of the hCG intron (FIG. 2): the intron was inserted between the nucleotides 1,761 and 1,762 of the LacZ coding sequence (pCMVβ). The gene cassette was then split at the intron (TGC↓AGGGCC), and was separately cloned and packaged into two AAV viral vectors. Two split AAV vectors were co-infected into the target cells. The two viral vectors were rejoined at the ITRs by recombination to form a head-to-tail heterodimeric DNA molecule. Two primers, PCR-F (5′-CTGGATCAAATCTGTCGATCCTTCCCGCCC-3′ (SEQ ID NO: 3)) and PCR-R (5′-CTGCTGCTGGTGTTTTCGTTCCGTCAGCGC-3′ (SEQ ID NO: 4)), were used to characterize the heterodimeric DNA and its mRNA transcript. As shown in FIG. 1d, large pre-mRNA was made using the heterodimeric DNA as the transcription template. The intron and ITR sequence were present in the ‘pre-mRNA’. FIG. 2 shows that maturation of mRNA by splicing leads to the removal of the intron along with the ITR, thus, restoring the original open-reading frame. FIG. 2F shows that the correct large protein was made as a final product.

Although split AAV vector viral particles must be used to test for heterodimerization, plasmids were used initially as the substrates to evaluate the Intron approach for the restoration of LacZ. A comparative study was conducted using three different LacZ expression plasmids (parental plasmid CMV-LacZ; CMV-LacZ-Int, containing the hCG intron in the LacZ gene; and CMV-LacZ-Int-ITR, containing an ITR inserted in the intron) to test the intron functionality with or without the ITR insertion. The plasmid construct with the ITR inserted in the intron should mimic the identical substrate predicted from viral heterodimer formation (FIG. 2 and 2D). X-gal staining and β-galactosidase enzyme activity assay after transfection into 293 cells showed no substantial difference between the three plasmids (β-galactosidase enzyme activities, in unit/mg protein: CMV-LacZ, 1.51×10⁴; CMV-LacZ-Int, 3.38×10⁴; and CMV-LacZ-Int-ITR, 1.32×10⁴). Thus, the presence of an intron element nearly doubled the expression of LacZ, whereas insertion of the ITR within the intron abrogated this increase. Thus, plasmid transfection provided independent confirmation that the intron strategy would work.

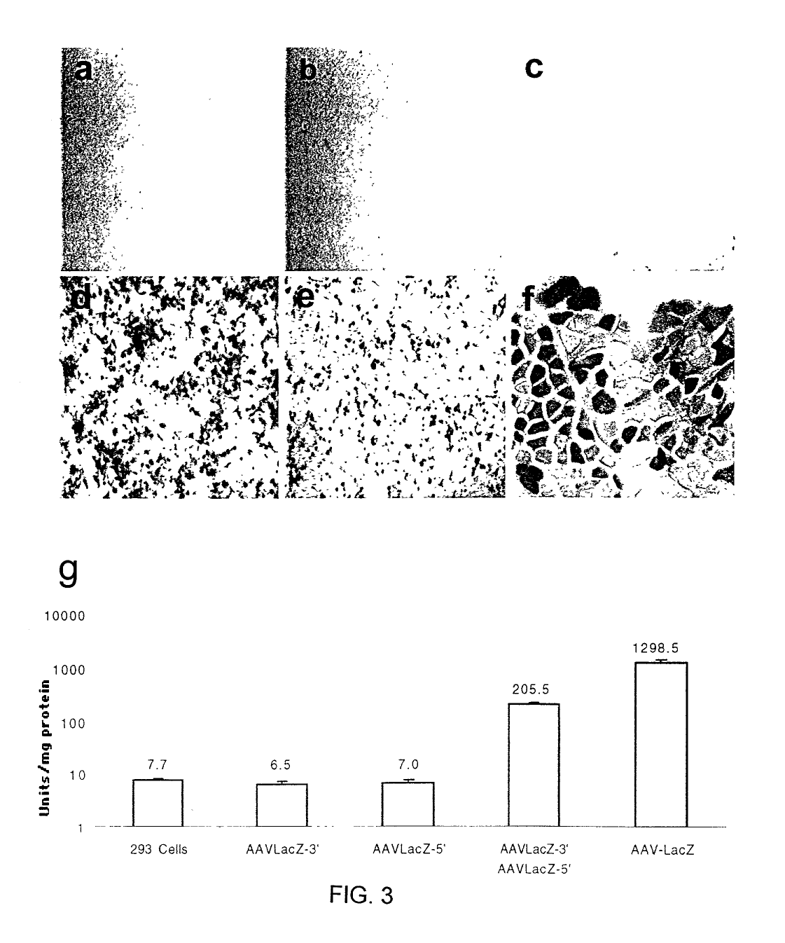

It is preferred in this approach that each split vector alone must be deficient in generating a functional RNA transcript or protein molecule. This deficiency was ensured by the lack of a poly(A) signal in one vector and the lack of a promoter in the other (FIG. 2B). To confirm that the split-gene vectors were negative for functional LacZ until heterodimer formation occurred, AAV vector plasmids containing the split LacZ were transferred into 293 cells and assayed for β-galactosidase expression. No blue cells were detected by X-gal staining when plasmids pAAVLacZ-5′ or pAAVLacZ-3′ were transfected individually. Whereas the CMV-LacZ control plasmid transfection led to strong X-gal staining in about 80% of the cells (indicative of transfection efficiency), a co-transfection of the two split-vector plasmids showed only few scattered blue-stained cells (FIG. 3c), with levels of β-galactosidase enzyme activity barely above background (13.1 units/mg protein and 7.7 units/mg protein in un-transfected 293 cell control). This result indicates that correct recombination indeed occurred between the two circular plasmid substrates but was very inefficient.

To demonstrate efficient restoration of the split LacZ gene both in vitro and in vivo by the two split AAV viral vectors through heterodimerization, high-titer AAVLacZ-5′ and AAVLacZ-3′ viral particles were produced using the adenovirus-free, ‘triple-transfection’ method of Xiao, et al.,1998, which is incorporated herein by reference in its entirety. Co-infection of AAVLacZ-5′ and AAVLacZ-3′ in 293 cells with an equal multiplicity of infection of 5×10³ DNA particles/cell led to extensive LacZ expression, and more than 50% of the cells became blue, with varying intensity, by X-gal staining at 4 days after infection (FIG. 3e). Infection by a single ‘un-split’ AAV-LacZ control vector at the same multiplicity of infection generated even more-intense LacZ blue staining (FIG. 3d). However, individual split-vector infections generated no blue cells and no LacZ expression (FIG. 3a and 3b). Moreover, in vivo injection of AAVLacZ-5′ and AAVLacZ-3′ viral particles simultaneously into the hind-leg muscles of mice yielded efficient and long-term (4 months) expression of LacZ (FIG. 3f).

The results described above strongly support the idea that heterodimerization is the mechanism for restoration of LacZ expression, and that the inter-molecular recombination is much more efficient by viral DNA than by circular plasmid DNA. A preliminary quantitative comparison showed that at a multiplicity of infection of 5×10³ viral particles and at 4 days after infection, the β-galactosidase enzyme activities generated from the single ‘un-split’ vector (205 units and 1,298 units, respectively; FIG. 3g), indicating that the split vectors were less efficient than the ‘un-split’ vector in gene expression. In addition, restoration of LacZ activities was also dependent on the ratio and the concentration of the two split AAV vectors (data not shown).

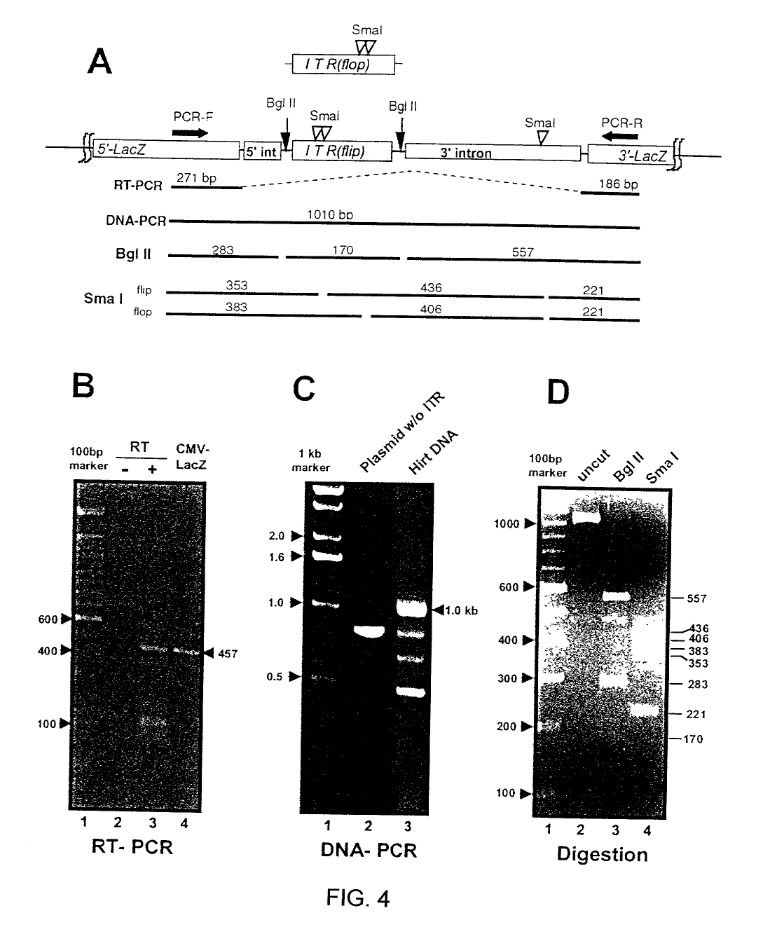

To confirm the β-galactosidase expression data that supported the idea of heterodimerization by AAV vectors, the rejoining of the split LacZ gene was investigated using both RNA and DNA analyses. First, RT-PCR with primers located on two separate vectors was used to determine whether correct mRNA was made. PCR-F is a forward primer located near the ‘right’ terminus of AAVLacZ 5′ vector, whereas PCR-R is a reverse primer located near the ‘left’ terminus of AAVLacZ-3′ vector (FIGS. 2C and 4A). RT-PCR using mRNA isolated from the cells co-transfected with the double vector generated a 457-base-pair (bp) fragment (FIG. 4B, lane 3), which was expected if correct vector joining and RNA splicing occurred (FIG. 4A). PCR of mRNA without prior reverse transcription generated negative results (FIG. 4B, lane 2). A 457-bp fragment also was obtained using plasmid CMB-LacZ (FIG. 4B, lane 4) as a positive control that lacks the intron in the coding sequence. No un-spliced product (1.0 kb) was detected, indicating that the removal of the intron and ITR by RNA splicing was efficient.

In addition, DNA PCR analysis was conducted in low-molecular-weight DNA isolated by Hirt extraction method from the double vector-infected cells. This ‘Hirt DNA’ should contain various forms of the viral vectors within the cells, including the heterodimers. As shown in FIG. 4A, a 1.0-kb fragment was obtained with PCR using Hirt-extracted DNA (FIG. 4C, lane 3). The control plasmid CMV-LacZ-Int, which lacks the ITR in the intron, generated a shorter (840-bp) PCR product (FIG. 4C, lane 2). These data are indicative of viral DNA heterodimerization through the ITR sequences. Additional PCR products other than the main 1.0-kb band (FIG. 4C, lane 3), were detected, which might be derived from the aberrant recombination products, such as head-to-head vector dimer junction (about 450 bp) and the head-to-tail dimer junction that lost the ITR (about 840 bp). To confirm the presence of the head-to-tail heterodimer junctions, restriction enzyme analysis was performed on the 1.0-kb PCR product that was purified by agarose gel electrophoresis (FIG. 4D, lane 2). Digestion with both BglII and SmaI indicated the presence of the AAV ITR structure, and the ITR was flanked by the intron sequence (FIG. 4D, lanes 3 and 4). Digestion with Small also confirmed the presence of ITR in both the ‘flip’ and ‘flop’ (FIG. 4A) orientations, and has also demonstrated many variant ITR structures (data not shown), which might be the result of ITR recombination and/or ligation.

Described herein is a heterodimerization strategy to overcome the size limitation of AAV vectors; this nearly doubled the packaging capacity of AAV by the mechanism of viral DNA heterodimerization. Although dimerization of the two split viral vectors was rather efficient (FIG. 3E), the recombination between the two split plasmid vectors was extremely inefficient (FIG. 3C). This phenomenon mirrors the disparity between the integrating efficiencies of AAV viral vectors and transfected AAV plasmid vectors. This may be attributed to the structural differences between viral and plasmid vectors. One is single- stranded and linear with ITRs at the termini, and the other is double-stranded and circular, indicating that the single-stranded viral DNA is more recombinogenic. The heterodimerization strategy has been used successfully in vitro and in vivo with a split LacZ gene cassette, thus proving the principle.

### EXAMPLE 2

Split AAV Vectors (SAVE) With Becker-Form Dystrophin Gene

Background

Duchenne muscular dystrophy (DMD) is a common and clinically devastating muscle disease, with a worldwide incidence of one in 3,500 male birth. DMD is caused by a defective dystrophin gene (cDNA larger than 11 kb) that fails to produce functional dystrophin. Some patients with very mild forms of Becker muscular dystrophy (BMD) express smaller forms of dystrophin due to in frame deletion within the dystrophin gene. A mini dystrophin gene from one of the patients has been cloned as a 6.3 kb cDNA. It produces a smaller but still functional dystrophin, and was only slightly less effective than the full length. Transgenic studies showed that this 6.3 Becker-dystrophin gene can almost completely prevent the development of dystrophic symptoms in transgenic mdx mice.

Duchenne muscular dystrophy (DMD) is the most common form of X-linked muscular dystrophy, with a world-wide incidence of one in 3,500 male births. DMD patients appear normal until 3-5 years of age, when they begin to experience progressive muscular weakness, starting with large proximal skeletal muscles. The typical affected individual is wheelchair-bound by the age of 12 and succumbs to cardiac or respiratory failure in the mid to late 20s. Becker muscular dystrophy (BMD) is a milder form of DMD with delayed onset and longer life span. Most DMD/BMD cases are transmitted via an unaffected mother (heterozygote), whereas 30% of cases have no previous family history and are considered to be due to a de novo mutation in the germ line of either the mother or her parents.

DMD and BMD are caused by a defective dystrophin protein in a patient's muscle cells. Dystrophin is a large protein of 3,685 amino acids (aa) and has three structurally distinct regions.

The N-terminal region is 136 aa long and forms a globular domain. The C-terminal region is 645 aa long and forms a second globular domain. The central region is a long and rod-like domain that consists of 24 repeats of a triple helical coiled-coil, or of 9 repeats in the smaller, but still functional, Becker form. The N- and C-terminal domains are separated, both in primary sequence and in tertiary structure by the central region. Each repeat is approximately 109 aa long, and there is 10-25% sequence identity between repeats. Each individual repeat is believed to fold independently into a structural module, and neighboring repeats are connected by a short, flexible linker sequence.

Dystrophin is a part of the dystrophin-glycoprotein complex and is thought to function by forming a submembrane lattice which enhances the tensile strength of the muscle membrane and by serving as an anchor for membrane proteins. The human dystrophin gene was identified in 1986 (Monaco, A. P., Neve, R. L., Colletti-Feener, C., Bertelson, C. J., Kurnit, D. M., and Kunkel, L. M. (1986), Isolation of candidate cDNAs for portions of the Duchenne muscular dystrophy gene, Nature 323, 646-650; incorporated herein by reference), the dystrophin protein was identified in 1987 (Hoffman, E. P., Brown, Jr., R. H., and Kunkel, L. M. (1987), Dystrophin: the protein product of the Duchenne muscular dystrophy locus, Cell 51, 919-928; incorporated herein by reference), and the complete dystrophin gene sequence (a 14-kbp cDNA) was cloned and determined by 1988 (Koenig, M., Hoffman, E. P., Bertelson, C. J., Monaco, A. P., Feener, C., and Kunkel, L. M. (1987), Complete cloning of the Duchenne muscular dystrophy (DMD) cDNA and preliminary genomic organization of the DMD gene in normal and affected individuals, Cell 50, 509-517; Koenig, M., Monaco, A. P., and Kunkel, L. M. (1988). The complete sequence of dystrophin predicts a rod-shaped cytoskeletal protein. Over 65% of the defective dystrophin genes found in DMD and BMD patients exhibit the loss (deletion) of one or more exons, with two deletion hot spots located in the 5′ end region and in the central region of the gene, resulting in smaller dystrophin protein. Deletions that cause frameshifts usually lead to DMD and are predicted to produce either a severely truncated dystrophin or no dystrophin at all.

Progression of the DMD and BMD disease cannot yet be slowed by therapeutic treatment. At present glucocorticoid administration is the only drug therapy, but it is only partially effective and has frequent side effects.

Alternative methods are being sought but have not yet been found. One potential approach is to upregulate the utrophin gene that encodes a close molecular analog of dystrophin. GAS However, it is impossible to predict if or when a nontoxic pharmacological agent would be identified to upregulate the human utrophin gene. Myoblast transplantation offers another potential treatment, but its clinical application has several limitations, including immunological problems, low spread and poor survival of the transplanted myoblasts. Another possibility is to convert DMD to BMD by correcting the frameshift mutation in the DMD dystrophin gene at the RNA level, but the necessary molecular tools remain to be found.

Gene therapy is recognized as the most plausible candidate for an effective therapy for DMD and BMD. A simple and proven way is to transfer into patient muscle cells a functional dystrophin gene that produces functional dystrophin. The endogenous defective dystrophin gene is harmless in the presence of the transferred functional dystrophin gene because DMD/BMD are recessive. A miniature version of the dystrophin gene, which was isolated from a BMD patient with very mild symptoms (England, S. B., Nicholson, L. V. B., Johnson, M. A., Forrest, S. M., Love, D. R., Zubrzycka-Gaarn, E. E., Bulman, D. E., Harris, J. B., and Davies, K. E. (1990), Very mild muscular dystrophy associated with the deletion of 46% of dystrophin, Nature 343, 180-182; Love, D. R., Flint, T. J., Sally, A. G., Middleton-Price, H. R., and Davies, K. E. (1991), Becker muscular dystrophy patient with a large intragenic dystrophin deletion: implications for functional minigenes and gene therapy, J. Med. Genet. 28, 860-864; both of which are incorporated herein by reference), can be used in the gene transfer. This minigene (6 Kbp cDNA) produces a smaller (1,983 amino acids, 200 kilo Daltons) but still functional dystrophin, which is only slightly less effective than the full-length dystrophin gene when tested in transgenic mdx mice. In efficiently transduced muscles of mdx mice, loss of force induced by lengthening contractions was alleviated.

Until now adenovirus-based vectors have been the most efficient vector for dystrophin gene transfer into muscles when tested on animal models (mdx mouse and dog) at an early age. However, adenovirus vectors have several important drawbacks. First, cellular and humoral immunity triggered by leaky expression of adenovirus proteins and input load of adenovirus proteins, respectively, eliminates transduced fibers and may enhance cytotoxic effects. Second, early non-immunological toxic effects reduce muscle force by approximately 20% and restrict the maximum adenovirus titer that can be used. Third, there is limited uptake of adenovirus into mature muscle fibers (compared to myoblasts), probably due to lack of cell receptors and the basal lamina barrier. Fourth, when directly injected into a limb muscle, adenovirus vector shows a moderate spread (1-3 mm), which necessitates a high concentration of injection sites. Adenovirus vector injected into the systemic venous circulation is mostly expressed in liver. New adenovirus vectors that are under development may partially overcome some of these drawbacks, but progress is limited.

Adeno-associated virus (AAV) vectors have characteristics that overcome the drawbacks associated with adenovirus vectors as applied to therapy for DMD. The safety of AAV vectors is discussed above. Additionally, AAV vector efficiently transduces mature muscle fibers. Also, when directly injected into muscle, AAV vector diffuses more broadly and rapidly due to its smaller particle size and due to its ability to bypass myofiber basal laminae and thereby transduce mature muscle cells.

Methods and Results

The Becker-form dystrophin gene (6.3 kb cDNA) was split at middle (GAGATCCCTGGAAG/GTTCCGATGATGCAGTCC (SEQ ID NO: 5)). The split site sequence (AG/GT) fits the consensus border sequence required for an intron splicing. The intron used for the split AAV vectors is human chorionic gonadotropin (hCG) gene 6 intron 1, as described above. The two dystrophin fragments were separately cloned into two individual AAV vectors, named as AAV-5′ Dys and AAV-3′ Dys. AAV-5′ Dys had a CMV promoter, a 5′ dystrophin fragment and a 5′ half hCG intron, but no polyA. On the other hand, the AAV-3′ Dys had a 3′ half-intron, a 3′ dystrophin fragment, and a polyA, but no promoter. After co-infection in 293 cells, these two AAV vectors formed heterodimers, and consequently restored the split-dystrophin gene, and produced functional dystrophin, as identified by western blot.

### EXAMPLE 3

Split-AAV Heterodimerization Construct for ATP-Binding Cassette (ABC1)

ATP-Binding Cassette (ABC1), a regulator of cellular cholesterol efflux, is approximately 7000 bp. Therefore, AAV cannot package the entire ABC1 sequence. To circumvent the problem, the ABC1 gene was split into two parts. A human chorionic gonadotropin (hCG) intron, as in Example 1, was inserted in the central part of the ABC1 coding sequence to act as a recognition site for recoupling of the two parts. Once the HCG-intron has rejoined during heterodimerization, it is excised during normal mRNA splicing removing the fused ITR sequence in the middle of the open reading frame and a complete ABC1 coding sequence is formed.

First, a parental plasmid, Myc,His,ABC (given to us by Dr. Jiang), carrying the mouse ABC1 gene (cDNA, GenBank Accession No. X75926) was digested with EcoRI and ClaI to excise an internal ABC1 fragment of workable size. The fragment was excised because the coding sequence of the ABC1 gene is over 6,500 nucleotides in length. The excised 980 bp fragment was gel purified and inserted by ligation into pBluescript II KS(+) (Stratagene, Inc. of La Jolla, California), also digested with EcoRI and ClaI. The newly formed plasmid, pBSKS-ABC1, was used as a PCR template, along with two (tail-to-tail) primers purchased from Genosys (Forward primer: 5′-GCCGCACCATTATTTTGTC-3′ (SEQ ID NO: 6) and Reverse primer:5′-CTTGCCGGTATTTTAGCAGG-3′ (SEQ ID NO: 7)) to create a linearized pBSKS-ABC1 plasmid at the correct intron site. The insertion site used (immediately after nucleotide 3322, according to GenBank Accession No X75926) was formerly an intron site. Two hCG intron primers purchased from Genosys (Forward primer: 5′-GTAAGACTGCAGGGCCCC-3′ (SEQ ID NO: 1) and Reverse primer: 5′-CTGGGACAAGGACACTGCTTC-3′ (SEQ ID NO: 2)) were used to create by PCR a 352 bp hCG-intron from template plasmid, pBSKS-Hcg-Intron. The hCG-intron PCR product was kinased and gel purified and the pBSKS-ABC1 PCR product was also gel purified. The intron and the pBSKS-ABC1 PCR product were ligated to form plasmid pBSKS-ABC1-Int. Plasmid pBSKS-ABC1-Int was digested with EcoRI and Clal and the 1332 bp fragment was reinserted into the Myc,His,ABC backbone it had been formerly taken from at the EcoRI/ClaI site. The new Myc,His,ABC1-Int plasmid was used as a template for PCR production of the two separate halves of the ABC1-Int construct. A forward primer (5′-TCAGTTAAGGCTGCTGCTGT-3′ (SEQ ID NO: 8)) at site 49 in the ABC1 gene and a reverse primer (5′-CCAACATTTCAGATCCGCAC-3′ (SEQ ID NO: 9)) in the middle of the HCG-Intron were used to produce the upstream or 5′ construct by PCR. The downstream, or 3′ construct was produced using a forward primer in the middle of the HCG-Intron (5′-GGTATCTCAGGTCCTCTGGG-3′ (SEQ ID NO: 10)) and a reverse primer at the end of the ABCI sequence (5′-ATGACCACTTAAAGGACCTG-3′ (SEQ ID NO: 11)). Both the upstream and the downstream constructs were approximately 3.5 Kb in length. The constructs were then kinased and gel purified. The upstream construct was inserted into pXX-UF1-CB digested with SalI made blunt with Klenow fragment in a manner that operably linked the CB regulatory sequences to the upstream construct, thereby constructing the upstream ABC1 vector (Plasmid pXX-UF1-CB is a derivative of pXX-UF1, described in Li et al., “rAAV Vector-mediated Sarcoglycan Gene Transfer in a Hamster Model for Limb Girdle Muscular Dystrophy,” Gene Therapy 6, 74-82 (1999), incorporated herein by reference in its entirety, in which the CMV promoter is replaced by a regulatory sequence including the CMV enhancer linked with a 227 bp nucleotide fragment containing the chicken β actin promoter). The downstream construct was inserted into pXX-UF1 that had been digested with KpnI and SalI and was made blunt with T4 DNA polymerase, thereby constructing the downstream vector. The upstream vector has the promoter to drive the gene and the downstream vector has the polyA; therefore, neither AAV, alone, can form a functional gene until heterodimerization occurs. (Sun, L., Li, J. & Xiao, X., “Overcoming adeno-associated virus vector size limitation through viral DNA heterodimerization,” Nature Medicine 6:599-602 (2000), incorporated herein by reference in its entirety).

### EXAMPLE 4

Split AAV Heterodimerization Construct for Factor VIII

Hemophilia A, classic hemophilia, is caused by a deficiency in Factor VIII. Human Factor VIII is synthesized as a preprotein of 2351 amino acids, which is processed to yield an active heterodimer protein. Human Factor VIII is well characterized. Factor VIII has been successfully transferred in vivo and expressed by AAV vectors. Different from those described herein (see, U.S. Pat. No. 6,200,560, describing human Factor VIII biology and rAAV Factor VIII vector systems).

An AAV heterodimer construct for Factor VIII, and, alternatively, for a B-domain deleted version of Factor VIII, may be constructed, as follows. A series of dual vector cassettes may be constructed substantially as described in Example 3. The coding sequence of Human Coagulation Factor VIII (GenBank Accession No. M14113, nucleotides 172 to 7227) is split into two segments, an upstream or 5′ segment and a downstream or 3′ segment, of roughly equal size, and preferably at a junction between exons. The sequence of the B-domain deleted Factor VIII also may be split in the same manner. The B-domain deleted Factor VIII is described in Chao, H., et al., “Sustained Expression of Human Factor VIII in Mice Using a Parvovirus-based vector,” Blood 95, 1594-1599 (2000), incorporated herein by reference in its entirety.

A 5′ vector is constructed by standard methods, such as those described above, to carry two AAV ITR sequences flanking the CMV promoter linked to the 5′ segment and a 5′ splice junction sequence. The 3′ vector is constructed to carry two AAV ITR sequences flanking the 3′ segment linked to splice acceptor and 3′ splice junction sequences and a polyadenylation signal sequence. Recombinant AAV particles for the upstream and downstream vectors were prepared according to standard three-plasmid transfection method, as described above. 293 cells were transduced by the rAAV particles and head-to-tail dimerization was determined by PCR. Production of the Factor VIII gene product was detected by ELISA. AAV viral particles containing both the upstream construct and the downstream construct were injected into the portal vein of mice and Factor VIII gene product was detected in the plasma of the mice for over four months.

Those of ordinary skill in the art will appreciate that various changes in the details, methods and materials which have been herein described and illustrated in order to explain the nature of the invention may be made by the skilled artisan within the principle and scope of the invention as expressed in the appended claims.

We claim:

1. A recombinant AAV virus particle, comprising a vector comprising two adeno-associated virus inverted terminal repeat sequences flanking a nucleic acid sequence comprising one of:
(a) a promoter operably linked to a first expressed nucleic acid having at its 3′ end a 5′ intron splice junction;
(b) a second expressed nucleic acid operably linked to a polyadenylation sequence and having at its 5′ end a 3′ intron splice junction operably linked to a splice acceptor sequence; and
(c) a third expressed nucleic acid having at its 5′ end a 3′ intron splice junction operably linked to a splice acceptor, and at its 3′ end a 5′ intron splice junction.

2. The virus particle of claim 1, said virus particle comprising capsid proteins and/or rep proteins of AAV types selected from the group consisting of AAV type 1, AAV type 2, AAV type 3, AAV type 3B, AAV type 4, AAV type 5, AAV type 6, avian AAV and ovine AAV.

3. The virus particle of claim 1, wherein said inverted terminal repeats are of AAV types selected from the group consisting of AAV type 1, AAV type 2, AAV type 3, AAV type 3B, AAV type 4, AAV type 5, AAV type 6, avian AAV and ovine AAV.

4. The virus particle of claim 1, wherein said expressed nucleic acid encodes at least a portion of a protein selected from the group consisting of β-galactosidase, dystrophin, ABC1 and Factor VIII.

5. The virus particle of claim 1, wherein the promoter is the CMV promoter.

6. The virus particle of claim 1, wherein said splice junction and said splice acceptor sequences are splice junction and splice acceptor sequences of human chorionic gonadotropin gene 6 intron 1.

7. The virus particle of claim 1, the vector comprising two adeno-associated virus inverted terminal repeat sequences flanking a nucleic acid sequence comprising a promoter operably linked to a first expressed nucleic acid having at its 3′ end a 5′ intron splice junction.

8. The virus particle of claim 1, the vector comprising two adeno-associated virus inverted terminal repeat sequences flanking a nucleic acid sequence comprising a second expressed nucleic acid operably linked to a polyadenylation sequence and having at its 5′ end a 3′ intron splice junction operably linked to a splice acceptor sequence.

9. A pharmaceutical composition comprising a virus particle as claimed in claim 1 and a pharmaceutically acceptable excipient.

10. A nucleic acid vector comprising:
two adeno-associated virus inverted terminal repeat sequences flanking a heterodimer nucleic acid sequence spliced gene comprising one of:
(1) a promoter operably linked to a first expressed nucleic acid having at its 3′ end a 5′ intron splice junction;
(2) a second expressed nucleic acid operably linked to a polyadenylation sequence and having at its 5′ end a 3′ intron splice junction operably linked to a splice acceptor sequence; and
(3) a third expressed nucleic acid having at its 5′ end a 3′ intron splice junction operably linked to a splice acceptor, and at its 3′ end a 5′ intron splice junction.

11. The nucleic acid vector of claim 10, wherein said inverted terminal repeats are of AAV types selected from the group consisting of AAV type 1, AAV type 2, AAV type 3, AAV type 3B, AAV type 4, AAV type 5, AAV type 6, and avian AAV and ovine AAV.

12. The nucleic acid vector of claim 10, wherein said expressed nucleic acid encodes at least a portion of a protein selected from the group consisting of β-galactosidase, dystrophin and Factor VIII.

13. The nucleic acid vector of claim 10, wherein said promoter is the CMV promoter.

14. The nucleic acid vector of claim 10, wherein said splice junction and splice acceptor sequences are splice junction and splice acceptor sequences of human chorionic gonadotropin gene 6 intron 1.

15. The nucleic acid vector of claim 10, comprising two adeno-associated virus inverted terminal repeat sequences flanking nucleic acid sequence comprising a promoter operably linked to a first expressed nucleic acid having at its 3′ end a 5′ intron splice junction.

16. The nucleic acid vector of claim 10, comprising two adeno-associated virus inverted terminal repeat sequences flanking a nucleic acid sequence comprising a second expressed nucleic acid operably linked to a polyadenylation sequence and having at its 5′ end a 3′ intron splice junction operably linked to a splice acceptor sequence.

17. A method for preparing a recombinant adeno-associated virus particle comprising the steps of:
(a) providing a nucleic acid vector, said nucleic acid vector comprising two adeno-associated virus inverted terminal repeat sequences flanking a gene portion comprising one of:
(1) a promoter operably linked to a first expressed nucleic acid having at its 3′ end a 5′ intron splice junction;
(2) a second expressed nucleic acid operably linked to a polyadenylation sequence and having at its 5′ end a 3′ intron splice junction operably linked to a splice acceptor sequence; and
(3) a third expressed nucleic acid having at its 5′ end a 3′ intron splice junction operably linked to a splice acceptor sequence, and at its 3′ end a 5′ intron splice junction; and
(b) packaging the nucleic acid into a recombinant adeno-associated virus particle.

18. The method for preparing a recombinant adeno-associated virus particle of claim 17, the adeno-associated virus comprising capsid proteins and/or rep proteins of AAV types selected from the group consisting of AAV type 1, AAV type 2, AAV type 3, AAV type 3B, AAV type 4, AAV type 5, AAV type 6, avian AAV and ovine AAV.

19. The method for preparing a recombinant adeno-associated virus particle of claim 17, wherein the inverted terminal repeats are of AAV types selected from the group consisting of AAV type 1, AAV type 2, AAV type 3, AAV type 3B, AAV type 4, AAV type 5, AAV type 6 and avian AAV and ovine AAV.

20. The method for preparing a recombinant adeno-associated virus particle of claim 17, wherein the expressed nucleic acid encodes at least a portion of a protein selected from the group consisting of β-galactosidase, dystrophin, ABC1 and Factor VIII.

21. The method for preparing a recombinant adeno-associated virus particle of claim 17, wherein the promoter is the CMV promoter.

22. The method for preparing a recombinant adeno-associated virus particle of claim 17, wherein the splice junction and splice acceptor sequences are splice junction and splice acceptor sequences of human chorionic gonadotropin gene 6 intron 1.

23. The method for preparing a recombinant adeno-associated virus particle of claim 17, wherein the nucleic acid comprises a promoter operably linked to a first expressed nucleic acid having at its 3′ end a 5′ intron splice junction.

24. The method for preparing a recombinant adeno-associated virus particle of claim 17, wherein the nucleic acid comprises a second expressed nucleic acid operably linked to a polyadenylation sequence and having at its 5′ end a 3′ intron splice junction operably linked to a splice acceptor sequence.

25. A method for producing a spliced ribonucleic acid comprising the step of transducing a cell capable of transduction by a recombinant adeno-associated virus particle with:
(a) a first virus particle containing a nucleic acid vector comprising two adeno-associated virus inverted terminal repeat sequences flanking a nucleic acid sequence comprising a promoter operably linked to a first expressed nucleic acid and having at its 3′ end a 5′ intron splice junction;
(b) a second virus particle containing a nucleic acid vector comprising two adeno-associated virus inverted terminal repeat sequences flanking a nucleic acid sequence comprising a second expressed nucleic acid operably linked to a polyadenylation sequence and having at its 5′ end a 3′ intron splice junction operably linked to a splice acceptor sequence; and
(c) optionally, a third virus particle containing a vector comprising two adeno-associated virus inverted terminal repeat sequences flanking a nucleic acid sequence comprising a third expressed nucleic acid having at its 5′ end a 3′ intron splice junction operably linked to a splice acceptor, and at its 3′ end a 5′ intron splice junction.

26. The method for producing a spliced ribonucleic acid of claim 25, wherein the cell is selected from the group consisting of central nervous system cells, muscle cells, lung cells, gut cells, liver cells, and eye cells.

27. The method for producing a spliced ribonucleic acid of claim 25, wherein the inverted terminal repeats are of AAV types selected from the group consisting of AAV type 1, AAV type 2, AAV type 3, AAV type 3B, AAV type 4, AAV type 5, AAV type 6 avian AAV and ovine AAV.

28. The method for producing a spliced ribonucleic acid of claim 25, wherein the expressed nucleic acid encodes at least a portion of a protein selected from the group consisting of a β-galactosidase, dystrophin, ABC1 and Factor VIII.

29. The method for producing a spliced ribonucleic acid of claim 25, wherein the promoter is the CMV promoter.

30. The method for producing a spliced ribonucleic acid of claim 25, wherein the intron splice junction and splice acceptor sequences are splice junction and splice acceptor sequences of human chorionic gonadotropin gene 6 intron 1.

31. The method for producing a spliced ribonucleic acid of claim 25, wherein the first expressed nucleic acid and the second expressed nucleic acid together encode a complete LacZ protein, the promoter is the CMV promoter, and the 5′ and 3′ intron splice junctions and splice acceptor sequences are sequences of human chorionic gonadotropin 6 gene intron 1.


SEQUENCE LISTING (11 sequences)

SEQ ID NO: 1 - DNA, 25 bp, Artificial Sequence
  misc_feature (1)..(25): hCG gene 6 intron 1 forward PCR primer
gtaagactgcagggcccctgggcac

SEQ ID NO: 2 - DNA, 24 bp, Artificial Sequence
  misc_feature (1)..(24): hCG gene 6 intron 1 reverse PCR primer
ctgggacaaggacactgcttcacc

SEQ ID NO: 3 - DNA, 30 bp, Artificial Sequence
  misc_feature (1)..(30): Forward PCR primer for detection of heterodimerization
ctggatcaaatctgtcgatccttcccgccc

SEQ ID NO: 4 - DNA, 30 bp, Artificial Sequence
  misc_feature (1)..(30): Reverse PCR primer for detection of heterodimerization
ctgctgctggtgttttcgttccgtcagcgc

SEQ ID NO: 5 - DNA, 32 bp, Homo sapiens
  misc_feature (14)..(15): intron insertion point after nucleotide 14
gagatccctggaaggttccgatgatgcagtcc

SEQ ID NO: 6 - DNA, 19 bp, Artificial Sequence
  misc_feature (1)..(19): Forward PCR primer for linearization of pBSKS-ABC1
gccgcaccattattttgtc

SEQ ID NO: 7 - DNA, 20 bp, Artificial Sequence
  misc_feature (1)..(20): Reverse PCR primer for linearization of pBSKS-ABC1
cttgccggtattttagcagg

SEQ ID NO: 8 - DNA, 20 bp, Artificial Sequence
  misc_feature (1)..(20): Forward PCR primer for 5′ ABC1-Int insert
tcagttaaggctgctgctgt

SEQ ID NO: 9 - DNA, 20 bp, Artificial Sequence
  misc_feature (1)..(20): Reverse PCR primer for 5′ ABC1-Int insert
ccaacatttcagatccgcac

SEQ ID NO: 10 - DNA, 20 bp, Artificial Sequence
  misc_feature (1)..(20): Forward PCR primer for 3′ ABC1-Int insert
ggtatctcaggtcctctggg

SEQ ID NO: 11 - DNA, 20 bp, Artificial Sequence
  misc_feature (1)..(20): Reverse PCR primer for 3′ ABC1-Int insert
atgaccacttaaaggacctg